(19) United States
(12) Patent Application Publication
(10) Pub. No.: US 2011/0056108 A1
(43) Pub. Date: Mar. 10, 2011

(54) RETROFITTED AND NEW WEAPONS WITH BIOMETRIC SENSORS FOR MULTIPLE USERS USING FLEXIBLE SEMICONDUCTORS

(76) Inventors: Jonas McCord, Beverly Hills, CA (US); Hans Schodel, Wessling (DE)

(21) Appl. No.: 12/128,475

(22) Filed: May 28, 2008

Related U.S. Application Data
(60) Provisional application No. 61/043,048, filed on Apr. 7, 2008.
(60) Provisional application No. 60/941,100, filed on May 31, 2007.

(51) Int. Cl. F41A 17/00; A61B 5/117

(52) U.S. Cl. 42/70.01; 40/633

(57) ABSTRACT

Semiconductors on a flexible substrate provide the sensing circuitry needed for personalizing a handgun grip. In one embodiment, a portion of the semiconductor structure can be applied to a surface using a technique similar to inkjet printing. The flexible semiconductor product can be applied to a sheet or mold placed over the grip, or into the injection molding of the grip itself. Alternately, the semiconductors can be initially formed and printed on a curved grip surface.

### CROSS-REFERENCES TO RELATED APPLICATIONS

This application claims the benefit of Provisional Applications No. 60/941,100, filed May 31, 2007, entitled “Retrofitted and New Weapons with Biometric Sensors for Multiple Users Using Flexible Semiconductors,” and. 61/043,048, filed Apr. 7, 2008, entitled “Smart Gun,” the disclosures of which are hereby incorporated herein by reference.

### BACKGROUND OF THE INVENTION

The present invention relates to personalized guns using biometric sensors, and in particular, to the retrofitting of guns with such sensors.

A number of technologies are designed to make guns safer by preventing accidental firing of the gun. One example is a drop safety which prevents the firing pin from striking the round if the gun is dropped. Another example is a magazine safety which prevents firing if the magazine is removed, even though a round has been left in the chamber. A safety can be activated by a lever which is moved, or in some instances, by squeezing the grip of the gun.

Another type of safety mechanism is the personalizing of a gun. These devices only let the gun be used by the authorized users, as indicated by a number of means.

There are a number of reasons why personalized guns are desired. In some instances, a police officer may have the gun taken away during a struggle and used on the police officer. In homes, parents may want to have a gun, but not have it usable by children in the house. Additionally, if a gun is stolen, it would be preferable that the thief not be able to use the gun.

For example, U.S. Pat. No. 5,713,149 has a firearm with a keypad into which the user must punch a code. Obviously, this mechanism requires too much delay to use the firearm in an emergency situation, and also does not prevent the firearm from being used by an unauthorized user once the firearm is enabled.

A number of systems use fingerprint or voice recognition to enable a firearm. Examples are U.S. Pat. Nos. 5,603,179, 5,560,135, 5,570,528, and 5,459,957. U.S. Pat. No. 6,785,995 suggests the use of both a fingerprint and voice recognition.

Another type of personalization mechanism uses a transmitter to enable or disable the firearm. For example, U.S. Pat. No. 4,682,435 uses an RF transmission to disable the firearm. U.S. Pat. No. 5,168,114 shows an RF transmitter worn by the user and a receiver, which is on the firearm. The firearm will not be enabled unless the transmitter is close to the firearm. U.S. Pat. No. 5,924,232 describes off-weapon signaling unit for enabling the weapon, such as a watch, bracelet, or other item worn by an authorized user

Smith and Wesson has developed a gun using the recognition technology of Lumidigm. Light from a cavity in the gun grip is projected into the user's hand, and from the reflected light sensing circuitry can distinguish differences in pigmentation, amount of blood, etc. While this may not be as accurate as a fingerprint, the processing is simpler and thus faster. This is described in U.S. Pat. No. 6,711,843.

The New Jersey Institute of Technology (NJIT) has done research on a grip recognition technology which can recognize the different pressure points of a hand on the grip of a gun. This is described, for example, in U.S. Pat. No. 6,817,130 and U.S. Pat. No. 6,763,126.

There are many disadvantages which prevent personalized guns from being commercially viable. For example, the personalization technology must recognize the user very rapidly so that the gun can be used in an emergency situation. In addition, the recognition needs to be very accurate. Finally, the recognition technology would preferably be adaptable to retrofitting existing firearms, not just building them into new firearms.

### BRIEF SUMMARY OF THE INVENTION

The present invention uses semiconductors on a flexible substrate to provide the sensing circuitry needed for personalizing a handgun grip. In one embodiment, a portion of the semiconductor structure can be applied to a surface using a technique similar to inkjet printing. The flexible semiconductor product can be applied to a sheet or mold placed over the grip, or into the injection molding of the grip itself. Alternately, the semiconductors can be initially formed and printed on a curved grip surface.

In one embodiment, a novel enrollment station is provided. A user obtains a new gun having the printed sensor grip, or has an existing gun retrofitted. The user then takes the gun to an enrollment station. The circuit on the gun is connected with the enrollment station, either wirelessly or by a wired connection. The user grips the gun, and the sensor readings are sent to the enrollment station for processing, and then appropriate parameters are sent back to the gun to be stored in a memory of the gun.

In one embodiment, the sensors detect multiple biometric parameters of a hand. These include, for example, the pigmentation of the skin and the concentration of blood vessels in different areas of the hand, using reflected light at designated wavelengths. Additionally, pH sensors can detect the unique aspects of a person's sweat in miniscule amounts.

In other embodiments, multiple sensing technologies are used, such as using both pressure sensing and the light sensing of the user's skin similar to that developed by Lumidigm. Additionally, voice recognition could be incorporated with a voice recognition sensor. The processor can determine a desired reliability from the multiple technologies detected to enable the gun. In one embodiment, multiple individuals can be personalized to use a single gun. The system can also be programmed to reject certain individuals, to give even more security than relying on failure to detect the correct user. This can be used, for example, to sense the hands of children in a household who are to be denied access to the weapon.

### BRIEF DESCRIPTION OF THE DRAWINGS

FIG. 1 is a diagram of the retrofitting of a gun handle using a flexible circuit created using semiconductor inkjet printing techniques.

FIG. 2 is a block diagram of the electronic circuitry on the flexible circuit and in the gun grip.

FIG. 3 is a block diagram of an enrollment system according to an embodiment of the invention.

FIG. 4 is a block diagram of the enrollment station of FIG. 3 according to an embodiment of the invention.

FIG. 5 is a top-level functional block diagram according to an embodiment of the invention.

FIG. 6 is an overview of the main state machine according to an embodiment of the invention.

FIG. 7 is a flowchart overview of the enrollment process according to an embodiment of the invention.

FIG. 8 is a flowchart overview of the unlocking/locking process according to an embodiment of the invention.

FIG. 9 is a flowchart overview of the firing of the Smartgun according to an embodiment of the invention.

FIG. 10 is a block diagram of a handgun biometric system and Biometric Module according to an embodiment of the invention.

FIG. 11 is a diagram that illustrates the operation of the sensors according to an embodiment of the invention.

FIG. 12 is a flowchart that illustrates the various states of the detection and extraction (DEM) and matching and enabling (MEM) electronics according to an embodiment of the invention.

FIG. 13 is a diagram of an alternate embodiment with biometric sensors in a wrist band, which communicates with an unlock mechanism in a gun.

### DETAILED DESCRIPTION OF THE INVENTION

### Handgun Grip

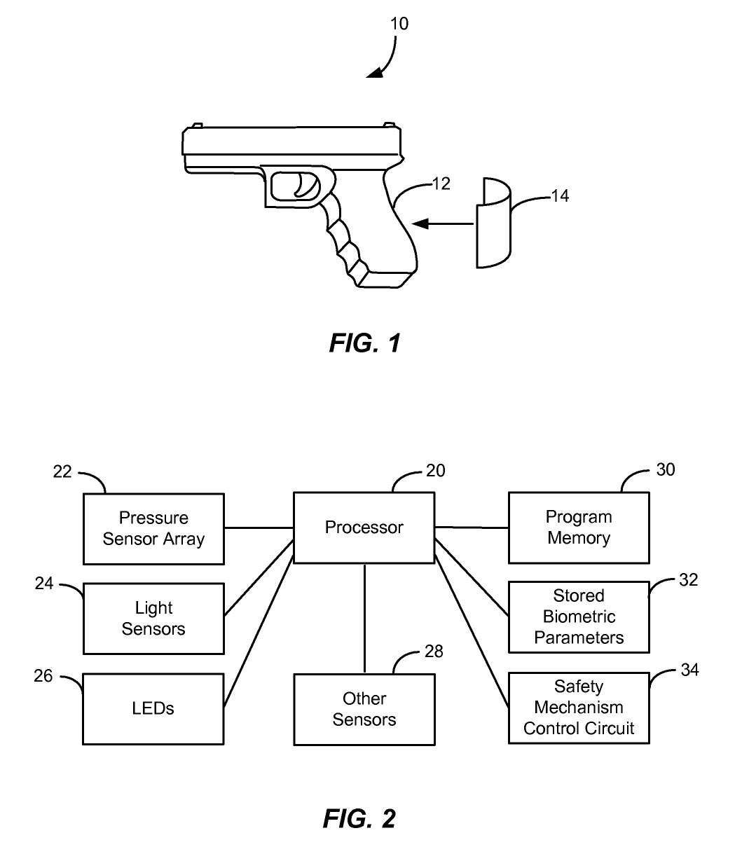

FIG. 1 shows a handgun 10 with a grip 12 onto which printed semiconductors for sensors, and optionally some control circuitry, can be printed. The printed semiconductors can be created on a flexible sheet 14, which wraps around the grip 12. Nanoident Technologies of Linz, Austria has a factory for manufacturing such inkjet printed circuitry. The sheet can be attached by bonding, lamination, or other techniques. Alternately, the printed semiconductors can be added to the mold for the grip. The printed circuitry could alternately be applied to a hollow handle grip which slides over the grip of a gun to retrofit it.

A number of techniques can be used to form semiconductors on a flexible substrate, with our without using printing. To achieve flexibility, in one embodiment plastic substrates are used instead of conventional glass substrates. When a plastic substrate is used, high temperature processing cannot be used but instead, low temperature processing is required. Accordingly, conventional silicon thin film transistors, which require high temperature processes for their formation, cannot be used. Alternately, organic semiconductors, which can be processed at low temperatures, are used in one embodiment. Portions of the transistors can be formed using organic semiconductor materials which can be applied using a printing method such as a ink jet method, a spin coating method, a spray method, a vapor deposition method, a dipping method or a casting method. In particular, in one embodiment the source and drain are formed using a resin paste containing metal powder. The source and drain are then connected using an organic semiconductor material which can be placed between the source and drain using an inkjet nozzle.

Examples of techniques for forming sensors using organic semiconductor materials on a flexible substrate are shown in Nanoident PCT patent applications with publication numbers WO 2006/26794, WO 2006/026795 and WO 2006/026796, the disclosures of which are incorporated herein by reference. Other examples of organic semiconductor techniques are set forth in US patent application publication No. 2007/0102699, No. 2006/0108579 and No. 2006/0216872, the disclosures of which are incorporated herein by reference.

Other examples of inkjet printing technologies for semiconductors are described, for example, in U.S. published Patent Application No. 20060251886, No. 20070102699, and U.S. Pat. No. 7,198,885, the disclosures of which are incorporated herein by reference. Such techniques have been adapted for providing, among other things, display screens.

A connection is made between the sensing circuitry and a circuit board mounted in the handle grip of the weapon. The circuit board includes a processor which generates the control inputs for any number of disabling mechanisms for the firearm. One example of such a disablement mechanism is shown in Springfield, Inc. U.S. Pat. No. 6,691,445. That mechanism includes a body positionable to block movement of the hammer spring assembly in order to prevent the hammer from being moved to its cocked position.

FIG. 2 is a diagram of the circuitry, a portion of which is printed onto the handle of gun 10 of FIG. 1. A processor 20 is manufactured in a standard manner, and mounted in the grip and connected to the printed circuitry. The printed portion includes a light sensor array 24 and LEDs 26. Also, ph sensors and other sensors can be included. Additionally, in one embodiment, a pressure sensor array 22 is included. The pressure sensor array can be, for example, that described in U.S. Pat. No. 6,817,130 for detecting the unique pressure of a user's hand. The individual array elements can be spread throughout the handle grip, interspersed with other circuitry for controlling the sensors. A memory is connected to the processor, although in one embodiment memory cells can be included in the printed circuitry. Light sensor 24 can be positioned adjacent to LEDs 26 to provide the optical sensing capabilities, such as that described in U.S. Pat. No. 6,711,843. Thus, in a preferred embodiment, only the sensing circuitry is printed on the grip, with a chip containing the processor and memory being attached to the disabling mechanism in the grip or mounted elsewhere on the gun.

Other sensors 28 can also be applied, such as a microphone element and voice recognition circuitry. A program memory 30 is provided, along with a memory 32 for storing the biometric parameters of the approved persons for using the gun. Finally, safety mechanism control circuitry 34 is provided for activating the particular safety mechanism controlled by the printed sensor system.

### Enrollment System

In one embodiment, an enrollment system is also provided. A user who has a gun retrofitted, or buys a new personalizable gun, can go to one of a number of enrollment stations, which may be located in a bank, in kiosks (like ATMs), in gun stores, etc. The user puts a hand on the grip of the weapon, and the sensor outputs are read by the microprocessor, and then sent to a processor in the enrollment station. The communication with the enrollment station can be wireless or wired. For a wired connection, a port may be provided in the grip which can be connected to a cable attached to the enrolment station. The act of connecting notifies the processor in the grip to send the data. For a wireless connection, the processor in the grip can detect and synch with the enrollment station, such as in the same manner that a wireless laptop computer detects and connects to a wireless network. The enrollment station processor examines the sensor data and prepares parameters to be stored in the memory of the grip for later use in detecting the same authorized hand.

In one embodiment, the user is required to present appropriate identification to the enrollment station as well. The information is then sent over the Internet to a service center for matching to the authorized owner and other authorized users of the gun. Alternately, the authorization information can be downloaded to the enrollment station, and a local comparison can be made.

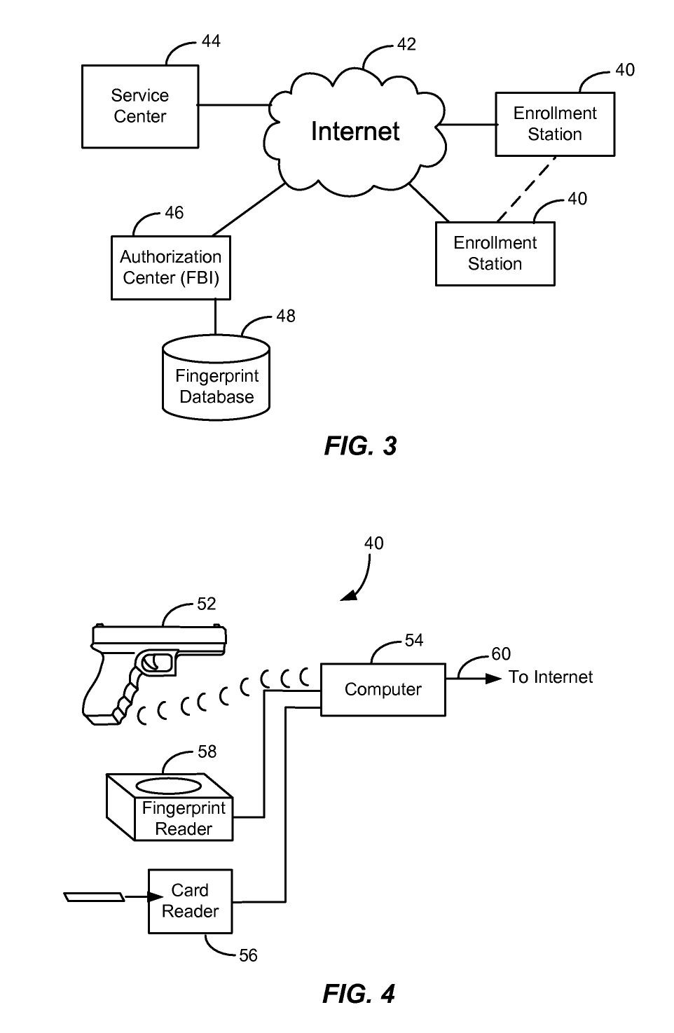

FIG. 3 is a block diagram of an embodiment of an enrollment system according to the invention. An enrollment station 40 is used to collect biometric information from a user, and user I.D. information. Multiple, distributed enrollment stations are used in one system. The ID information is sent over the Internet 42 to a service center 44. The service center verifies the user, and sends a message back to the enrollment station. The enrollment station then reads the sensors, calculates the appropriate parameters, and stores them in the memory of the gun. The enrollment station doesn't store the parameters, thus insuring privacy of the biometric information. In other embodiments, the biometric information could be locally stored, or sent to a secure remote database. The enrollment station can be located in a kiosk, bank, gun store, or can even be sold to users for home use by connecting it to the Internet.

As part of the enrollment process, the user identification information can be provided to an authorization center 46, such as the Federal Bureau of Investigation (FBI), which can compare user I.D. information, including fingerprints, to a database 48 of known criminals. Even if this check has already been done at the time of gun purchase, this enables the checking of additional users.

FIG. 4 is a diagram of one embodiment of an enrollment station 40 according to the system of FIG. 3. A gun 52 has a grip which is grasped by a user and includes sensors for detecting the user's particular biometric parameters. These parameters are then sent to a computer 54 in the enrollment station via a wired or wireless connection. Additionally, the user may be identified using a card reader 56 which may, for example, read credit card information or other information of a user. Additionally, the card reader could include a keyboard for the user to input social security or other identifying information. Finally, a fingerprint reader 58 is optionally provided for the user to provide a fingerprint which can be compared to the fingerprint provided when the gun was purchased, and/or to the FBI fingerprint database. In one embodiment, the data collected is sent to the Internet over a connection 60.

According to one embodiment of the invention, the printing technologies of Nanoident, set forth in published PCT applications may be used. These include PCT Application Nos. WO2006/026794, No. WO2006/026795, and WO2006/026796.

In one embodiment, the printed circuit is connected to a battery mounted in the weapon to drive the mechanical disablement mechanism. The circuitry is powered down, and not activated unless someone who is authorized grips the weapon. Additionally, the battery is turned off so it will not be drained if there is no clip in the weapon.

In one embodiment, the enrollment station may provide the data to video games, such as in arcades. A user could pay for a number of games at the time or enrollment, or later over the Internet. Instead of a gun, the user grasps a gun-handle shaped grip with sensors that is attached to the enrollment station. Alternately, a half moon shaped grip with a generic hand reverse molded into the top could be used, or other shapes. Then all the user needs to do is grip the handle of an appropriate video game to be authorized to play and have the playing time deducted from the user's account. The video game handle can be programmed to accept any user enrolled. Alternately, the invention could be used for any device with a grip, such as power tools which parents don't want children using, or equipment in an industrial setting that only trained operators will be authorized to use. The same enrollment station can obtain sensor information from either its attached grip, or a gun handle or other device grip connected to it, wired or wirelessly.

In one embodiment, the enrollment stations may be used to provide biometric identification information for systems other than guns. For example, handgrips could be provided at checkout counters for point-of-sale devices instead of having to use a credit card. Instead of a hand grip, a flat palm reader may be used. An example of such a system using fingerprints is described at www.paybytouch.com, an IBM partner.

The sensing technology of the invention could be applied to other products as well. For example, it could be used to certify consumer electronics, designer clothes, etc. A retail store could read the biometric imprint on a tag, and compare it with the imprint sent by the manufacturer over the Internet to verify that it is genuine. The biometric imprint can be of a checker or other person at the manufacturer.

In one embodiment, the printed semiconductor technology may be applied to new handles and integrated with a disabling mechanism in the handle of the gun. In alternate embodiments or retrofitting, the gun needs to be retrofitted to add the mechanical disabling device. Such a disabling device will prevent a bullet from being loaded into the chamber unless an authorized person grabs a weapon.

In one embodiment, the printed circuitry may be embedded into a glove worn by the user, with the glove providing an activation signal to the gun. The processing circuitry is imbedded in the gun. The flexible semiconductor sensors can be mounted inside the glove, and connect with circuitry in the grip either wirelessly (e.g., Bluetooth or WiFi), or contacts on the outside of the glove can make contact by simple pressure with contacts on the grip. Also, a battery in the grip can power the sensing circuitry in the glove through capacitive coupling, a direct connection, induction or otherwise.

### Detailed Module Description

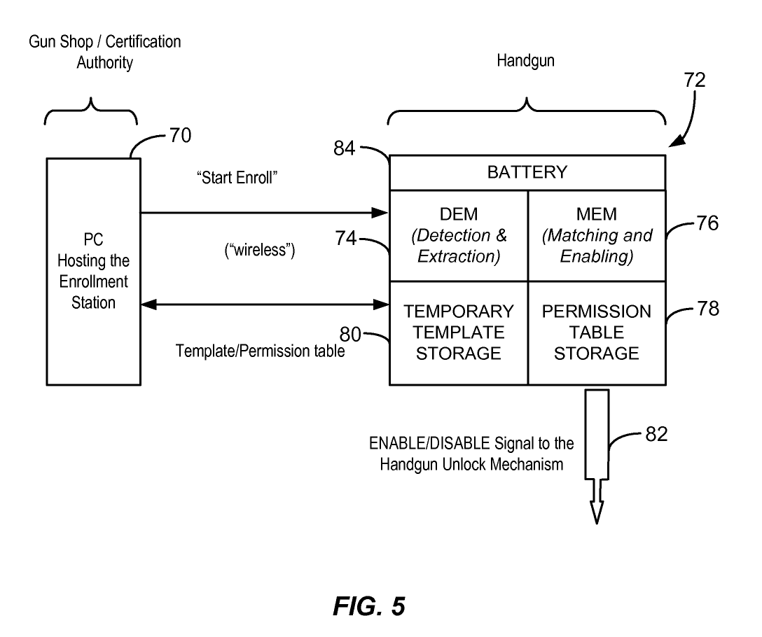

A handgun system is shown in FIG. 5 according to an embodiment of the invention includes:
1. An enrollment station 70, to enroll and to entitle a user (or multiple users of the same group) to the use of a smartgun.
2. An authentication system 72 referred to as the Biometric Module that verifies the identity of a user (possibly a member of a larger group) and that provides the enabling/disabling signals to the unlock mechanism.

The enrollment station 70 consists of:
1. Software for the man-machine interface to help the Certification Authority or its representative to perform the enrollment of users.
2. A wireless electronic interface between the Biometric Module and the Enrollment PC, so as to enable communication between the Enrollment PC memory and the Biometric Module during the enrollment phase and/or the maintenance operations.

The Biometric Module 72 consists of a set of electronic blocks:
1. A Detection and Extraction Module (DEM) 74 that is basically a “biometric key generator” whose purpose is: (1) To detect the physical connection of the hand-grip with the user's hand; (2) To extract the biometric template of the user intending to use the Smartgun.
2. A Matching and Enabling Module (MEM) 76 whose purpose is: (1) To discriminate an individual (possibly member of a larger group) who intends to use the Smartgun by comparing the biometric template captured by DEM (in a temporary template storage 80) to the templates of authorized users stored in the Permission Table 78; (2) To deliver an ENABLE or DISABLE instruction 82 to an Unlock Mechanism (UM).
3. A Battery Module (BAT) 84 to power up the DEM, MEM and the UM electronics and an Actuator/Unlock Mechanism.

The “biometric key” is a unique non-transferable identifier associated to one single person and acquired from a biometric detection system located in the hand-grip. The discrimination will be performed within the Smartgun itself against a Permission Table containing both (1) The ID of the Smartgun, and (2) the templates of the users and/or of the groups that are entitled to use the Smartgun. This data will be loaded into the Smartgun during the enrollment phase in presence of a Certification Authority or its representative in a gun shop. The discriminator will be able to differentiate between multiple users or group ID.

The “Detection and Extraction” are processes consuming a lot of power, which are required to run continuously. In order to minimize the power consumption of the Biometric Module and to increase the time between 2 successive maintenance operations, in one embodiment, a switch on the Smartgun powers on/off the Biometric Module. This switch is activated by the safety mechanism of the Smartgun. Alternatively, a capacitive or other hand detection switch can be used.

### Enrollment Process

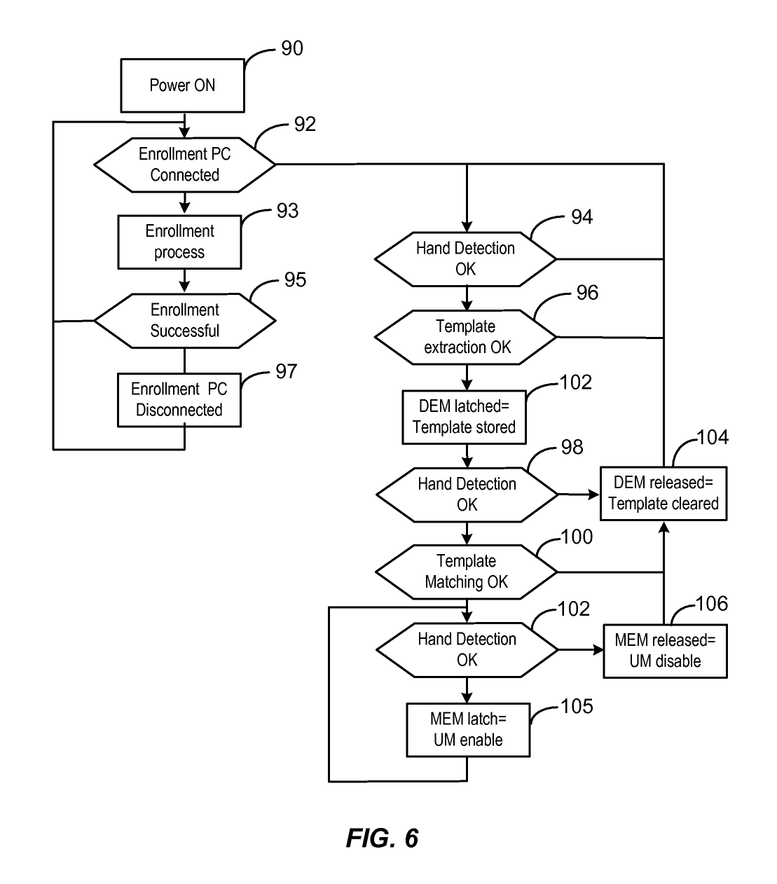

A complete enrollment process is illustrated in the state machine of FIG. 6. The owner (and users authorized by the owner) will be enrolled in the weapon shop in presence of a Certification Authority or its representative. To do so, the Biometric Module is turned on (90) and is connected temporarily (92) to the Enrollment PC, such as via a wireless connection. Then the biometric data of the enrolling user will be captured through the biometric sensor of the Biometric Module, and then the unique features of the biometric data will be extracted, ciphered and transferred in the form of a template to a memory embedded within the Biometric Module; and/or to the Enrollment PC hard disk (93). Once the authorized user has been enrolled (95), he is then entitled to use the Smartgun, and the enrollment PC is disconnected (97).

### Enrollment Phase

The enrollment of the weapon owner will take place at the gun shop in presence of an Authority entitled to certify the identity of the user and the ownership of the Smartgun. The biometric data of the Smartgun owner will be captured by the Biometric Module and the biometric features (template) will be stored in a non-volatile memory embedded in the Biometric Module and/or on the Hard Disk Drive (HDD) of the Enrollment PC or alternatively in a central data base. During the enrollment and subsequent visits to the gun shop, the Smartgun owner will have the ability to authorize other users to use the Smartgun. Each authorized user will have to enroll at the gun shop in presence of the owner and of the Authority.

For a new user, a new entry with the Smartgun ID will be created in the Permission Table located inside the Smartgun. After a successful enrollment, the user template will then be transferred and stored in the entry just created in the Permission Table. For all subsequent enrollments or un-enrollments, only the data relevant to users to be added to or removed from the Permission Table will be transferred.

To commence an enrollment process, a Certification Authority or its representative first activates the wireless link between the Smartgun and the Enrollment PC. Then the user is requested to hold on to the Smartgun (94). The enrollment will be initiated by the authorized Agent from the Enrollment PC. A message will be shown on the display of the Enrollment PC indicating that the template extraction (96) has been completed. Then the authorized Agent will request the user to hold on to the hand-grip one more time (98) and initiate a verification process. Once the verification is completed, the result (100) will be shown on the display of the Enrollment PC as well as indicated via the LED on the Smartgun (described below).

Depending on the result, the authorized Agent can decide to keep and save the template (102) or restart the process again (104). Regardless of the verification result, the authorized Agent may repeat the enrollment process to add more templates or just as a training process for the user to get familiar with the usage of the Smartgun. It should be noted that the more templates are added to the user template, the higher the False Acceptance Rate (FAR) will be for this particular user. It should also be noted that the more verifications (trainings) are done during the enrollment process, the more familiar the user will be in using the Smartgun, hence the lower the False Rejection Rate (FRR). Once training is completed, the user's hand can be detected (102) to either enable the unlocking mechanism (105) or disable it (106).

### Template Extraction

The template extraction phase will be initiated from the Enrollment PC by sending the <<start enroll>> command to the DEM of the Biometric Module. Then, all the following actions will be completed by the Biometric Module: (1) The biometric data of the palm (or part of it) is captured; (2) The biometric features are extracted and the corresponding template is generated; (3) The template generated is stored in the Biometric Module's non-volatile memory (Permission Table) and/or in the HDD of the Enrollment PC. The template extraction is considered as successful if the captured biometric data fulfills pre-defined quality criteria.

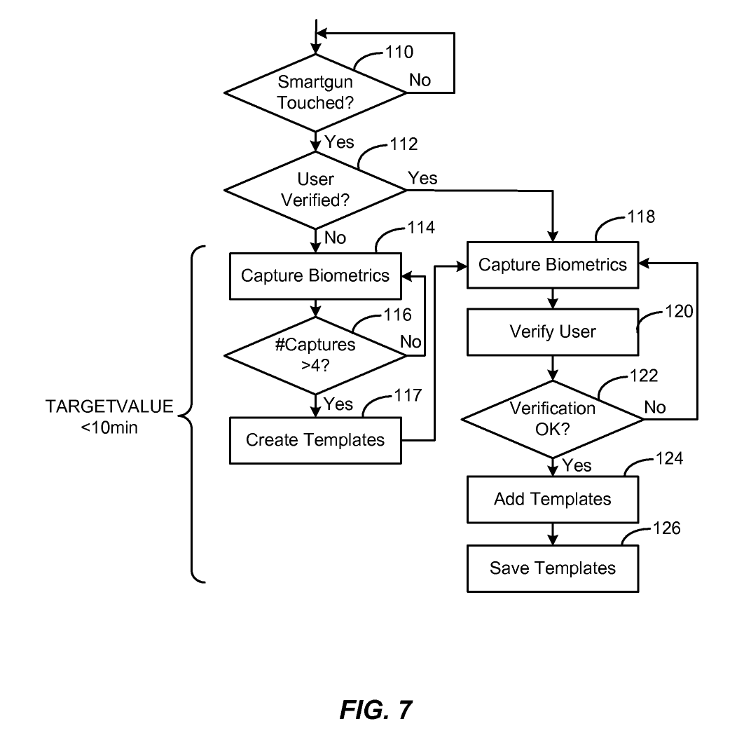

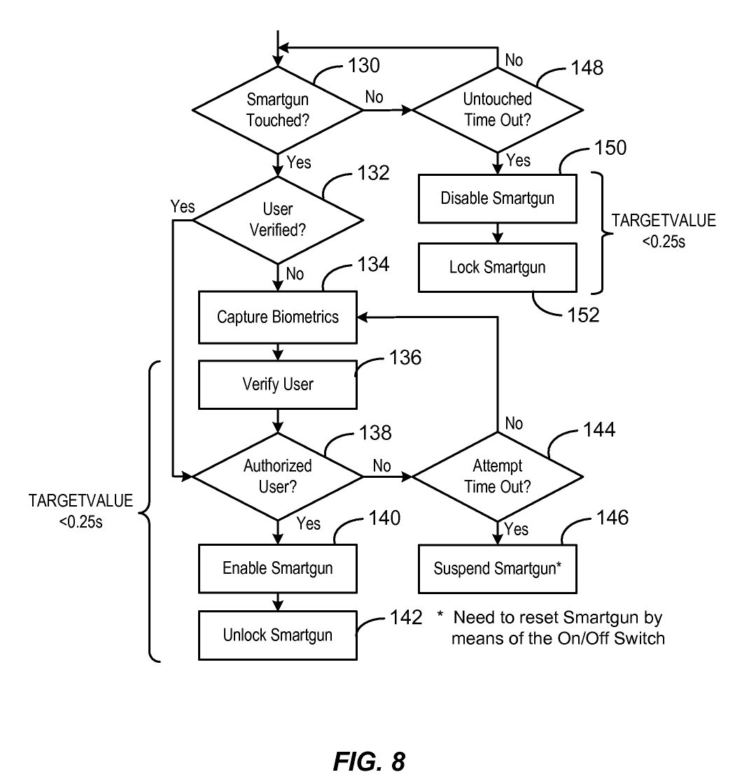

As shown in the flowchart of FIG. 7, the smartgun is first switched on (with on/safety switch) and touched (110). If the user is not verified (112), the biometrics are captured (114). This process is repeated for a desired number of captures, such as more than 4 (116). Once enough captures have been done, the templates are created (117). The templates are tested by comparing to another biometric capture (118) which if successful will verify the user (120). If the verification is not OK (122), the process is repeated. If the verification is OK, the templates are added (124) and stored (126).

### Template Verification

Immediately after the template extraction, the user will be requested to follow the complete verification process synopsis. The purpose of this is to verify that an enrolled user can be recognized effectively with the extracted biometric template. The following actions will be carried out sequentially: (1) The newly generated template is matched against the templates stored in the non-volatile memory; (2) Scoring policies and results are displayed onto the monitor of the Enrollment PC.

A specific LED located on the Biometric Module will provide feedback to notify the Certification Authority and/or the user if the enrollment process was successful or has to be re-started. When the enrollment has been successful completed, the authorized Agent may end the enrollment process and switch off the wireless connection to save power consumption.

### Biometric Handgun Operation

After the enrollment, the procedure to enable the use of the Smartgun is as follows:
1. The user must first power on the Biometric Module.
2. The user must hold the hand-grip.
3. The user biometric template must be properly extracted.
4. The user template must be successfully verified (against the Permission Table).
5. The hand of the identified authorized user must stay in contact with the hand-grip to keep the weapon enabled for use.

FIG. 8 is a flowchart overview of the unlocking/locking process. The template extraction will be made by the DEM as soon as the user holds the hand-grip (130). If the user has not already been verified after gripping (132), the biometrics will be captured (134) and the ciphered user template will then be sent to the MEM electronics of the Biometric Module to check whether the user is entitled to use the Smartgun. This involves verifying the user (136) and determining that the user is authorized (134), and not someone unauthorized (e.g., a child whose biometrics are recorded). The time required to make the full authentication of the user is the addition of the biometric pattern acquisition time plus the MATCHING TIME. If the user is recognized to be an authorized user (138), the ENABLE signal (140) will be sent to the Unlock Mechanism (142) and the Smartgun will be ready for use after a short time (the ENABLE TIME). A green/red LED will inform the user about the verification status. If there is no determination after a time-out period (144) that the user is authorized, the smartgun authentication operation is suspended (146) and it remains locked.

It is to be noted that because the False Rejection Rate (FRR) of any biometric system is in the range of a few percents, the complete extraction and matching process might have to be repeated. Consequently, this will increase the time before the user can effectively make use of the Smartgun (the delay will be at least the sum of the biometric pattern acquisition time, the MATCHING TIME and the ENABLE TIME+UNLOCKING TIME).

As long as the hand of an identified authorized user stays in contact with the hand-grip, the ENABLE signal will be sent constantly to the Unlock Mechanism to keep the weapon unlocked so the user will be able to fire at wish. When the hand is no longer in contact with the hand-grip after some “Time-Out” period (148) then the DISABLE signal (150) will be sent to the Unlock Mechanism and the Smartgun will be locked (152) after a very short time referred to as the DISABLE TIME. Once the Smartgun is disabled, the complete detection, extraction and matching process would have to be started over again.

Even if the user has been effectively identified, the conditions that could cause the disable and lock of the Smartgun are: (1) The hand is no longer in contact with the hand-grip; or (2) The battery level is too low. The user may switch off the Biometric Module after use to save power and maintain reasonable battery lifetime.

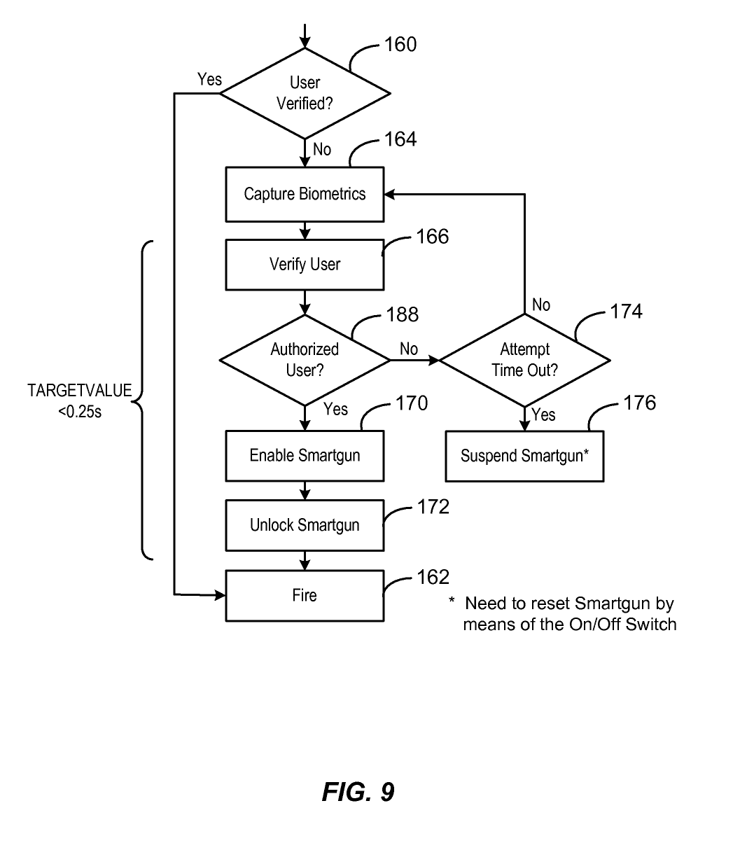

FIG. 9 is a flowchart overview of the firing of the Smartgun. If a user is already verified (160), the gun can be fired (162). If the user is not verified, the biometrics are captured (164), the user is verified (166), and the user is determined to be authorized (168). The gun is then enabled (170) and unlocked (172), and can be fired (162). If the user is not determined to be authorized after a time-out period (174), the smartgun is suspended (176).

### Hardware Architecture

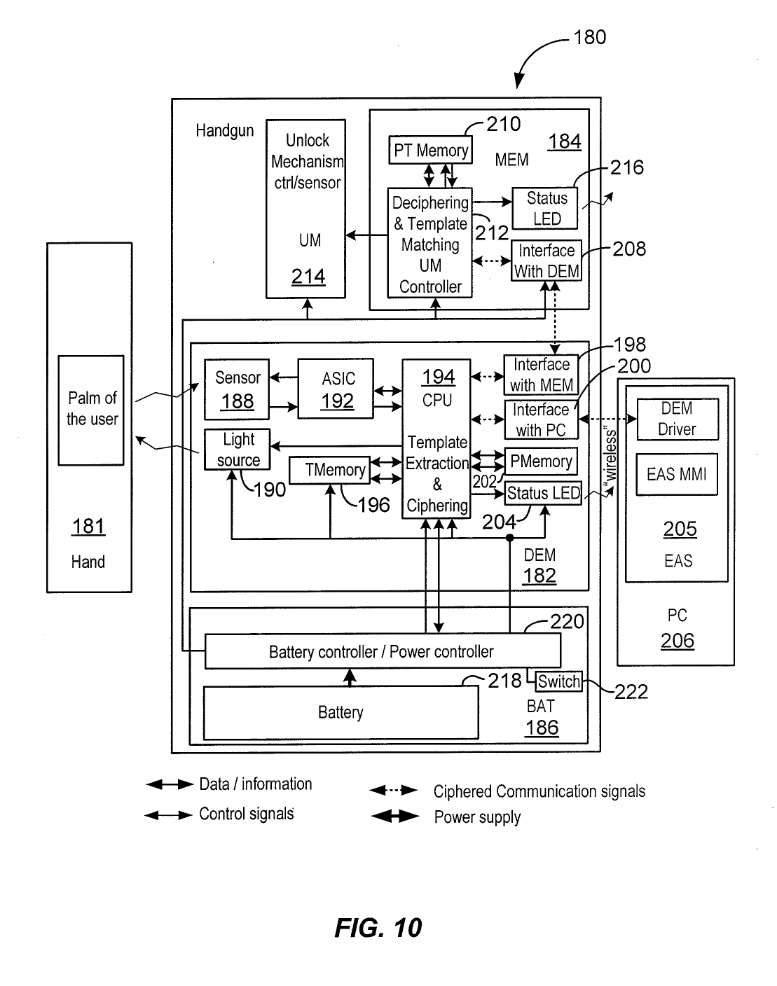

The handgun biometric system is composed of The Biometric Module (BM) and The Enrollment Application Software (EAS). FIG. 10 is a block diagram of the hardware architecture. The BM 180 includes The Detection and Extraction Module (DEM) 182, The Matching and Enabling Module (MEM) 184, and The Battery Module (BAT) 186.

DEM electronics extracts the user biometric template. It detects the presence of the hand 181 on the hand-grip and performs the necessary acquisition of the biometric data, the extraction of the biometric features, the creation, ciphering and storage of the templates.

The DEM consists of the following elements:
A biometric sensor to capture the biometric data [sensor 188 and light source 190].
Optionally, an additional sensor to detect the contact of the hand to the biometric sensor and to the hand-grip.
A sensor signal conditioning electronics [ASIC 192].
A microcontroller 194 or microprocessor based electronics to execute the biometric template extraction algorithm and to cipher the “biometric key” using template memory 196 and pattern memory 202 before sending it to the MEM [CPU and P/T Memory].
A wired communication system to exchange data with MEM [Interface 198 with MEM].
A low power bidirectional wireless communication link 200 to exchange data with the EAS (Enrollment Application System) that runs on the Enrollment PC 206 [Interface with PC] during the enrollment phase; Such a low power wireless system should also be present on the Enrollment PC.
LED indicators to show to the user the system status [Status LED 204].

MEM electronics performs the template matching and verifies if the user is entitled to use the Smartgun and send ENABLE/DISABLE signals to the Unlock Mechanism accordingly.

The MEM consists of the following elements:
A wired communication system to exchange data with DEM [Interface with DEM 208].
The Permission Table (in the form of a non-volatile rewritable memory 210) containing the ID information and the templates of the users who are authorized to use the Smartgun. The non-volatile memory must be able to support multiple users (TARGET VALUE: 11 users) [PT Memory].
An electronics module or controller 212 that matches and checks if the identified user is authorized to use the Smartgun and then sends an ENABLE/DISABLE signal to the Unlock Mechanism 214 accordingly [Verification Ctrl].
A status LED 216.

The BAT consists of:
A battery 218 to power the complete Biometric Module and the Unlock Mechanism;
A power management system 220;
A switch 222 to turn on/off the Biometric Module before and after each operation (the mechanical switch to activate the Smartgun will be used to trigger the power-on and power-off sequence of the Biometric Module).

A low power and low speed bidirectional wireless solution is used on both ends (Smartgun and Enrollment PC).

### DEM Description

The DEM is an embedded electronic system in the hand-grip aiming at detecting and extracting the user biometric data. The DEM also serves as the enrollment hardware device when connected to a PC through wireless interface to enable communication with the enrollment software.

The DEM system is based on a sensor that captures the biometric information and a template processor that generates the biometric template. This template is ciphered by the processor and then temporarily stored in a volatile memory before being transferred to the MEM through a dedicated interface.

The DEM is a portable system powered by a battery with a power management system to control the power consumption.

### Sensor Description

The biometric sensor is optimized in correlation with the dedicated light sources to capture the biometric data of the palm with appropriate techniques.

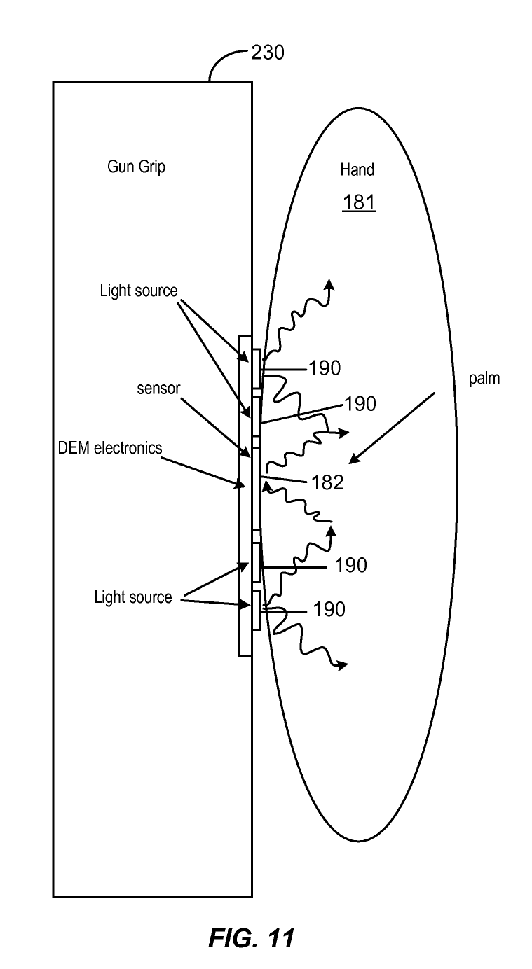

The sensor, in one embodiment illustrated in FIG. 11, is a matrix sensor on a gun grip 230 making direct contact with the skin surface. The matrix includes the DEM electronics 182 with the sensor, with interspersed light sources 190. The emitted light wavelength range will be carefully chosen to ensure correct propagation and diffusion into the tissues. It interacts with the user palm and is then captured by the sensor array. The sensor is an array of sensors interspersed with the light sources.

The sensor size and resolution optimizes:
The biometric performance (in conjunction with the authentication software)
The ease of the enrollment process for the user
The amount of data to be processed and stored
The power consumption and the battery lifetime
The cost of the solution (processing power of the microcontroller or microprocessor, size of the memories to store the templates, etc.).

The wavelengths used for the light sources take the following into account:
The nature and level of the back-scattered and reflected light from the palm and human tissues,
The sensor characteristics.

### Template Processor Unit Description

The template processor unit is based on a microcontroller or a microprocessor (CPU) to carry out all the DEM tasks as follows:
DEM state machine and sequencer.
Peripherals initialization and configuration.
Power management.
Protocol for the MEM interface.
Protocol for the PC interface.
Volatile and non-volatile memory management.
Template extraction and ciphering.

During the template extraction phase, the CPU extracts specific patterns and/or minutiae of the palm, and generates a unique palm signature called a template. This template is ciphered and then transmitted via the MEM interface.

The CPU controls LEDS to provide feedback to the user and possibly to the Certification Authority during the enrollment phase.

LED for the user template status:
GREEN blinking=user template is OK.
RED blinking=user template is not OK.

### Communication and Interface Description

The DEM communicates with the MEM in operational mode through a dedicated serial electrical connection. The data to be exchanged between DEM and MEM is ciphered by the CPU.

The DEM also communicates with the EAS when connected to the enrollment PC. A low-power/low cost bidirectional wireless PC interface is used.

The wireless interface uses a specific driver so as to allow the EAS to address the DEM hardware.

This driver makes it possible:
To configure, initialize and address the internal registers of the DEM and MEM (Note: addressing MEM hardware from the Enrollment PC is achieved through the DEM so the user must keep in contact with the Smartgun throughout the enrollment process);
To read and write with a secured protocol the template data in the DEM memory;
To read and write with a secured protocol the permission list data in the MEM memory;
To verify and report the operation of DEM and MEM built-in test;

### MEM Description

In one embodiment, the MEM is physically separated from the DEM so it can be integrated within or nearby the Unlock Mechanism for security reasons.

The MEM consists of the matching processing unit that compares the template to the Permission Table stored in a non-volatile memory. Upon a successful authentication, the “ENABLE” state (authorization of use) is latched and transmitted to the Unlock Mechanism as long as the user's hand remains in contact with the hand-grip.

The matching result is shown directly to the user by a dedicated “LOCK STATUS” LED.

LED for the enrollment/authentication status:
GREEN=user authenticated and/or enrollment completed.
RED=user NOT authenticated.

### BAT Description

The handgun biometric system is a portable device with a reasonably long lifetime. To reach this target, all embedded electronics consume ultra low power. Functions are disabled or put into idle state as soon as they are no longer needed.

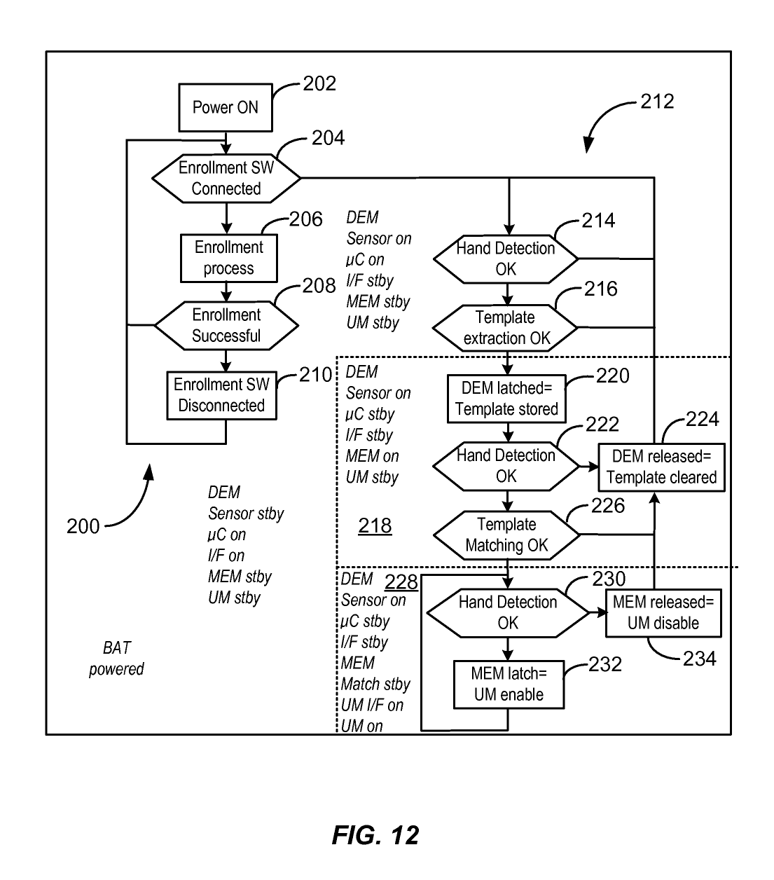

The various states of the Detection and Extraction Module (DEM) and Matching and Enabling Module (MEM) electronics and of their main constituents are described in the diagram of FIG. 12. In a first state 200, the DEM sensor is on standby, the microprocessor (uC) is on, the interface (I/F) is powered on, and the MEM and UM modules are on standby. The steps in this state are power on (202), enrollment software connected (204), enrollment process (206), enrollment successful (208), and enrollment software disconnected (210).

A second state 212 has the same conditions as the first state, except that the sensor is on, instead of in standby. The steps in this second state are hand detection OK (214) and template extraction OK (216). In a third state 218, the microprocessor and interface are put on standby, and the MEM is turned on. The steps are DEM latched=template stored (220), hand detection OK (222), template matching OK (226), and DEM released=template cleared (224). The fourth state 228 puts the MEM on standby, but the UM interface is on and the UM is on. The steps are hand detection OK (230), MEM latch=UM enable (232) and MEM released=UM disable (234).

### Authentication Software

The authentication software provides the necessary algorithm to extract minutiae and/or other biometric features from the biometric data captured by the biometric sensor. The authentication software is embedded in the Biometric Module and divided between the DEM and the MEM.

The authentication software supports multiple users (e.g., 11 users).

The registered owner is the “System Administrator” and is the only person who can authorize additional users to use the same Smartgun.

The enrollment will take less than 10 minutes. This is the time required to acquire, extract and store 5 templates for an untrained user.

### Wristband

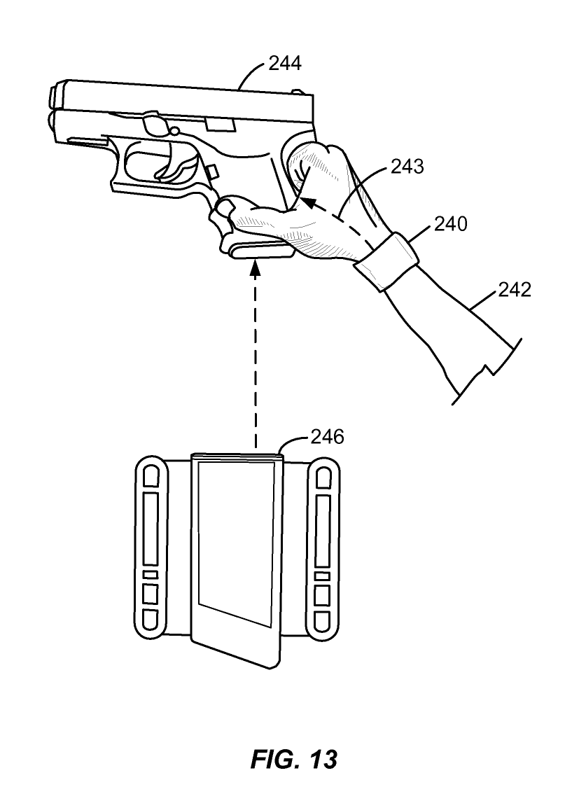

In one embodiment, the system can be divided into different modules which communicate with each other. For example, as shown in FIG. 13, the biometric sensors and comparison circuits can be mounted in a wristband 240. The sensors will identify the user based on the profile of the user's wrist, not the user's arm. Upon verification of an authorized user, a signal is sent through the skin of the users arm 242 on a path 243, where it is detected by a contact sensor in the handle of gun 244. The signal can be an authorization code, which matches a code stored in gun 244. Upon a match, the gun is unlocked. One example of transmitting electrical signals through the skin is set forth in Sony's US Published Application No. 20060252371, incorporated herein by reference.

An advantage of such a wrist sensor is that the guns can be interchangeable among multiple users. This can be important for police or soldiers, where it is desirable, for example, to pick up the gun of a fallen comrade and be able to use it. Also, it allows a larger group of users for a gun, where the template storage capability of a gun is limited. Hundreds or thousands of users could be authorized for a weapon.

The sensor array, or a sensor module, can be woven into fibers for the wrist band, a glove, or other wearable item. The sensor array would be programmed at an enrollment station for the user(s) of the wrist band or glove, and would recognize the user(s). Once the wrist band is on, and the user grips a biometric weapon, a signal would be sent authorizing the weapon to unlock. The authorization sensors are in the wrist band, and not the weapon, which would only receive a go or no go signal, a confirm or not confirm. This means a thousand users could pick up the same weapon and use it, since only a confirm signal is being sent by the person wearing the wrist band.

The wrist band sensor array is personalized to the user's skin under the wristband (or under the glove or other wearable item) similar to the way the sensor in the gun would work as described above. The user can select where to wear the band, and this area would be scanned similar to the way the palm is scanned with the enrollment station. The difference is the timing. As soon as a user puts on the wristband, the user is recognized as an owner of this band. In the wristband there is a CPU and memory storing the biometric templates. Through the surface of the skin this information's is transported to the gun or other articles the user is allowed to use. Since the wristband will already be on before the user picks up the weapon, there is no delay for user recognition when the weapon is picked up—the user has already been recognized. This technology is useful not only for guns, but anything else a user touches with the user's hand, such as a doorknob (for building access), a steering wheel, an airplane control, a mobile phone, a computer, etc. It can also be used to monitor the coming and going of people who use the sensor to gain entrance or access to a computer, or facility, or weapon system.

The wearable item can be used for access at a turnstile to an amusement park, a video game controller, or other things that require payment. The user's credit card can be linked to the users templates at enrollment, and a code identifying the user can be sent to the access object, in addition to the unlocking code. Multiple fields of a transmission can be used for multiple access devices, so a single wristband can be used for many purposes. To not compromise speed for weapon access, the first field can be for weapon activation. Alternately, multiple transmissions can be used, with the first for weapon activation. Alternately, the access device can identify itself to the wristband first, with the appropriate code then being returned.

The code transmitted to the weapon can be a common code for a group of authorized individuals. The wristband provides another degree of safety, similar to removing bullets from a gun. A child picking up the gun could not use it, and cannot be falsely recognized, without the wristband.

Where a glove is used instead of a wristband, there is no skin contact with the gun handle unless the glove has a hole in it. For such a glove without skin contact, since it will be in physical contact with the gun handle, an inductive or capacitive coupling could be used to transmit the unlock signal. Alternately, a near range transmission could be used, such as used in RFID tags.

The wristband, glove or other article has its own battery to power the sensor and matching circuitry. The battery can be rechargeable or replaceable.

Alternately, the wristband can send a wireless signal (e.g., Bluetooth) to a receiver in the gun to activate the gun. Since the biometric detection no longer needs to be done on the gun, a receiver chip could be mounted in a recess in the gun, and does not need to be on a printed circuit on the handle.

Alternately, a memory with the templates could be added to an ammunition clip 246 as shown in FIG. 13. The biometric sensors on the gun handle would detect the characteristics of the user's palm, and the CPU on the gun handle would compare them with templates in the memory of the gun clip. Insertion of the gun clip can provide an electrical connection between a contact on the clip, connected to the memory, and a contact to the circuit on the gun handle. Alternately, a CPU can be included in the clip as well. The clip (without the bullets) could be returned to a gun shop to be refilled with bullets. The owner can enroll at an enrollment station, and the gun shop can access the templates, over a secure Internet connection, and download them to the clip when the customer buys a clip.

### Alternatives

In one embodiment, if the template does not match the user's palm, shifted versions of the template are used to determine if the user's grip has shifted slightly. Matching algorithms similar to those used for motion compression (MPEG) are used.

In another embodiment, the user can control the degree of accuracy required for activation, within a predetermined range. For example, in a dirty environment, where there is likely to be dirt on the sensors or the user's hand, a more forgiving match can be allowed.

In one embodiment, the template of an unauthorized user trying to use the gun can be stored, and downloaded later to a PC or a charging station for the gun. This can then be used as evidence in a crime, such as where a criminal broke in, stole the gun, and tried to use it. In one version, the time of each use that enables the gun, or is unsuccessful, can be recorded for later review as evidence.

As described above, to save power and on/off switch can be tied to a safety mechanism for the gun. Alternately, a capacitive detection circuit can detect the proximity or touch of a hand, and turn on the power. In one alternative, once the gun has been enabled, it can power-down, but remain in an active state, for a time-out period. The period could be adjusted by the gun owner. For example, the user may want to activate the gun, then put a glove on in cold weather. The time-out period could be extended as long as a hand is detected present, even if it is a glove.

In one embodiment, a holster is provided for the gun that includes a battery charger. The holster has battery contacts at a position that matches contacts on the biometric circuit on the gun handle. Alternately, non-contact charging techniques can be used, such as through inductive coupling. The charger can also include a processor and memory, and can download and store recently recorded data on the gun. For example, the times of use, and the identity of the user or characteristics of an attempted user can be logged.

As will be understood by those of skill in the art, the present invention could be embodied in other specific forms without departing from the essential characteristics thereof. For example, a semiconductor inkjet printer includes a print head which directs semiconductor materials directly onto a handgrip or preformed, curved substrate. Alternately, instead of an enrollment station, the CPU of the gun could process the sensor readings when first activated, and store the parameters in its memory. A PIN or ID code might be required to allow such activation. Accordingly the foregoing description is intended to be illustrative, but not limiting, of the scope of the invention which is set forth in the following claims.

1. A personalized apparatus comprising:
a activation mechanism;
biometric sensors on a flexible substrate attached to a handle of said apparatus said biometric sensors sensing one of the pigmentation of the skin, the concentration of blood vessels and the pH of a person's sweat; and
a circuit, responsive to said biometric sensors, for controlling said activation mechanism.

2. The apparatus of claim 1 wherein said apparatus is a weapon.

3. The apparatus of claim 1 wherein a portion of said biometric sensors are printed on said substrate.

4. The apparatus of claim 1 wherein said circuit includes a memory storing a template of prerecorded characteristics of at least one authorized user.

5. The apparatus of claim 1 wherein said sensors are light sensors, and further comprising a plurality of light emitters for directing light of a limited wavelength range into the palm of a user.

6. The apparatus of claim 5 further comprising a plurality of pressure sensors.

7. The apparatus of claim 1 wherein said circuit is mounted on a circuit board separate from said flexible substrate, and electrically connected to said flexible substrate.

8. A personalized handgun comprising:
a activation mechanism;
biometric sensors on a flexible substrate attached to a handle of said handgun, wherein said sensors include light sensors,
a plurality of light emitters for directing light of a limited wavelength range into the palm of a user;
a memory storing a template of prerecorded characteristics of at least one authorized user; and
a circuit, responsive to said biometric sensors, for matching a pattern detected by said biometric sensors and comparing the pattern to said template, and controlling said activation mechanism in response to the comparison.

9. An enrollment station for providing personalized apparatus comprising:
an interface for obtaining biometric information from sensors on a handle of an apparatus;
a processor for generating parameters based on said biometric information; and
a memory coupled to said apparatus for storing said parameters for latter comparison, wherein said memory can store parameters for multiple users.

10. The enrollment station of claim 9 wherein said apparatus is a weapon.

11. The enrollment station of claim 9 wherein said processor is configured to download said parameters to a memory of said apparatus, said parameters identifying at least one authorized user.

12. A method for activating an apparatus comprising:
storing characteristics of a user's palm in a memory;
detecting the characteristics of the palm of a user with a plurality of biometric sensors on a flexible substrate attached to a handle of said apparatus;
comparing the detected characteristics to the stored characteristics; and
enabling the activation of said apparatus if said detected characteristics match said stored characteristics within a predefined margin of error.

13. The method of claim 12 wherein said apparatus is a handgun.

14. The method of claim 12 further comprising storing characteristics of a plurality of users' palms in said memory.

15. A personalized activation system comprising:
an access controlled object including
a activation mechanism for unlocking access to said object,
a memory for storing a unique authorization code, and
an input, communicatively coupled to said activation mechanism, for receiving an authorization code and comparing it with said stored authorization code, and in the event of a match, providing an activation signal to said activation mechanism; and
a wearable item including
biometric sensors on said wearable item mounted where the sensors will contact with the skin of a user,
wherein said biometric sensors can detect unique aspects of the skin of a user,
a memory storing a template of prerecorded characteristics of at least one authorized user, and
a circuit, responsive to said biometric sensors, for matching a pattern detected by said biometric sensors and comparing the pattern to said template, said template having an associated personal authorization code, and sending said personal authorization code to said input in response to the comparison.

16. The system of claim 15 wherein said input is a contact for receiving said activation signal through the skin of the user.

17. The system of claim 15 wherein said wearable item is a wristband and said object is a weapon.
